(19) United States
(12) Patent Application Publication
(10) Pub. No.: US 2015/0351886 A1
(43) Pub. Date: Dec. 10, 2015

(54) SURGICAL VAGINAL SUPPORT DEVICE FOR SURGICAL TREATMENT OF PELVIC ORGAN PROLAPSE

(71) Applicant: CAREY TASCA PTY LTD, Eltham, Victoria (AU)

(72) Inventor: Marcus Patrick Carey, Carlton, Victoria (AU)

(21) Appl. No.: 14/760,277

(22) PCT Filed: Jan. 16, 2014
(86) PCT No.: PCT/AU2014/000027

§ 371 Date: Jul. 10, 2015

Related U.S. Application Data
(60) Provisional application No. 61/753,716, filed on Jan. 17, 2013.

(51) Int. Cl. A61F 2/00; A61B 17/12; A61B 17/42

(52) U.S. Cl. CPC A61F 2/005; A61B 17/42; A61B 17/12099; A61B 17/12136; A61B 2017/12004

(57) ABSTRACT

A surgical vaginal support (SVS) device for use with surgical treatment of pelvic organ prolapse has a substantially horse-shoe shape, including an arched member which is curved distally. A transverse support member connects opposing side portions of the arched member toward the proximal end thereof. A cross member connecting proximal ends of the arched member side portions may include a region of reduced thickness and/or a lobe for grasping during removal. The SVS device may be adapted for use with a tamponading accessory, which may be inflatable.

### FIELD OF THE INVENTION

The present invention relates to surgical treatment of pelvic organ prolapse and in particular, to a vaginal support device for use with such surgical treatment.

### BACKGROUND TO THE INVENTION

Vaginal prolapse is a condition in which the bladder, uterus and/or bowel protrude into the vagina, typically due to loss of natural support for the pelvic organs and the vaginal vault in women who had undergone a prior hysterectomy. In the normal female anatomy, direct support for the vaginal vault is provided by the parametrium (cardinal and uterosacral ligaments) and paracolpium fibers. These fibers act like suspensory ligaments and arise from the fascia of the piriformis muscle, sacroiliac joint and lateral sacrum, and insert into the lateral upper third of the vagina. Indirect support for the vaginal vault is provided by the levator plate, formed by the fusion of the right and left levator ani muscles between the rectum and coccyx. Pelvic organ prolapse and vaginal vault prolapse occurs after failure of these direct and indirect supporting mechanisms and is frequently accompanied by weakness of the muscular pelvic floor and suspensory fibers of the parametrium and upper paracolpium.

In developed countries around one in nine women undergo surgery for pelvic organ prolapse. In the United Sates, more than 400,000 women undergo surgery annually for pelvic organ prolapse. Anterior and/or posterior colporrhaphy (native tissue repair) are the most commonly performed operations for pelvic organ prolapse. Sacral colpopexy is widely considered to be the gold standard operation for pelvic organ prolapse particularly in cases of recurrent vaginal prolapse and prolapse of the vaginal vault following hysterectomy. Vaginal vault prolapse occurs in approximately 10% of women following hysterectomy and occurs in equal numbers following abdominal and vaginal hysterectomy. In 2010 around 13% of women having surgery for pelvic organ prolapse in the United States underwent a sacral colpopexy procedure. The sacral colpopexy procedure can be performed through a laparotomy incision, laparoscopically or robotically. Many different vaginal, abdominal and laparoscopic procedures have been described to treat pelvic organ prolapse and there is currently no consensus on the most effective operation.

Dissatisfaction with native tissue repair (traditional colporrhaphy) for pelvic organ prolapse resulted in increased usage of mesh to augment vaginal repair procedures in order to obtain higher success rates. However, the use of mesh placed via a trans-vaginal incision during vaginal repair procedures is controversial. Studies have reported significant problems (e.g. pain, dyspareunia and mesh exposure) with the use of mesh during vaginal prolapse surgery. Recent FDA (Food and Drug Administration) warnings about the usage of trans-vaginal mesh have led to renewed interest in native tissue repair and sacral colpopexy.

Following surgery for pelvic organ prolapse, the repaired tissues are exposed to rises in intra-abdominal pressure as the patient mobilizes or with coughing, vomiting and straining with bowel evacuation. Rises in intra-abdominal pressure may adversely affect the healing of the vaginal repair procedure leading to surgical failure and recurrent prolapse. By supporting the vagina following surgery the risk of surgical failure and recurrent pelvic organ prolapse may be reduced.

To our knowledge a vaginal support device has only been used in the PROSIMA procedure (Ethicon, US). This surgery uses mesh to augment the prolapse surgery. Zyczynski et al reported the results of study conducted at 11 international centres evaluating the effectiveness at 1 year and the safety of the PROSIMA procedure (7). At 1 year, the leading edge of the vaginal wall was at cm above the hymen in 88.3% of the subjects and in 76.9% the leading edge was >1 cm above the hymen.

Based on global impression of change scores, 73.3% patients reported they were “much better” and 15.3% “a little better” at 1 year. All measures of QOL and sexual function improved significantly from baseline. At baseline, dyspareunia was reported in 13 / 62 (21.0%) sexually active patients; at 1 year, this was reduced to 7.7% (2 persistent, 3 de novo). Three patients (2.2%) underwent re-intervention for prolapse. The authors of this study concluded that prolapse surgery using the PROSIMA system was safe and resulted in good anatomical and functional outcomes at 1 year. However, a device to splint the healing vaginal tissues has not been used or studied as an adjunct to native tissue repair or sacral colpopexy.

Use of the vaginal support device used in the PROSIMA procedure has identified a number of drawbacks related to its design and shape. The trapezium shape is, in many cases, too wide at the top especially if a concomitant hysterectomy is performed (hysterectomy is frequently performed during vaginal surgery for pelvic organ prolapse). When a concomitant hysterectomy is performed there is frequently some narrowing at the vaginal apex and this is anatomically inconsistent with the trapezium shape of the PROSIMA vagina support device which widens toward its distal apex. Use of the PROSIMA vagina support device in these patients can cause distension of the vaginal wall at the distal corners of the device while providing inadequate support at the vaginal apex.

Use of the PROSIMA vagina support device and vaginal pessaries with and without a supporting membrane has led to the observation that the supporting membrane (both in the PROSIMA vagina support device and in vaginal pessaries that have a supporting membrane) is associated with increased vaginal discharge for patients utilising such devices post-surgery. Furthermore, the PROSIMA vaginal support device has ‘trimable’ sections at its apex so that the device may be modified to assume three different sizes. If a surgeon chooses to use a medium or large PROSIMA vaginal support device then either one or two of the trimable sections of the device remain in place. When a large or medium vaginal support device is used these additional sections are somewhat redundant and unnecessarily increase the bulkiness of the device resulting in increased vaginal discharge and discomfort for some patients in the post-operative period. The limitation to three potential sizes with the PROSIMA vaginal support device does not provide a sufficient range of sizes for the variation in vaginal dimensions following surgery for pelvic organ prolapse especially in cases of sacral colpopexy.

The discussion of the background to the invention included herein including reference to documents, acts, materials, devices, articles and the like is intended to explain the context of the present invention. This is not to be taken as an admission or a suggestion that any of the material referred to was published, known or part of the common general knowledge as at the priority date of any of the claims.

### SUMMARY OF THE INVENTION

Viewed from one aspect, the present invention provides a surgical vaginal support (SVS) device for use with surgical treatment of pelvic organ prolapse. The SVS device has a substantially horse-shoe shape, including an arched member which is curved distally and a transverse support member connecting opposing side portions of the arched member toward a proximal end thereof.

In a preferred embodiment, the SVS device includes a cross member in addition to the transverse support member. The transverse support member provides structural support to the device, by maintaining the opposing side portions of the arched member at an ideal separation for supporting the vaginal walls without distending them, during tissue healing after prolapse surgery. The cross member connects proximal ends of the arched member side portions and can be grasped by the surgeon, e.g. during removal of the SVS device from the vagina. To assist with grasping, the cross member may include a region of reduced thickness, ideally located substantially centrally of the cross member, providing a location for the surgeon to grasp the SVS device using the thumb and forefinger or a grasping device.

Additionally/alternatively, the cross member may include a lobe, tab or other proximally extending portion for grasping during removal of the SVS device from the vagina. The lobe/tab may be integral with the cross member. Alternatively, the lobe/tab may form a separate component of the SVS device which is temporarily or permanently affixed to the cross member.

In a preferred embodiment, the SVS device includes an opening through which at least part of a tamponading accessory may be received. In an embodiment having a transverse support member and a cross member, these are separated by a void, which provides such an opening. Typically, the tamponading accessory includes an inflatable body and inflation tubing. In use, the tubing is coupled at one end with the inflatable body, and with an inflation source at the other end. Ideally, part of the inflation tubing is received through the opening during preparation of the SVS device with the inflatable body for placement in the vagina. Once placed, the SVS device is retained in situ and the inflatable body is inflated, with part of the inflation tubing extending through the opening, e.g. using a syringe. The inflated body achieves tamponading, limiting post-operative bleeding.

Preferably, the SVS device includes one or more fastening features, such as suture eyelets. In a preferred embodiment, there is a suture eyelet provided on each lateral aspect of the SVS device, where the side portions of the arched member and the cross member meet. Additional suture eyelets may be provided e.g. for longer SVS devices. When the SVS device is correctly placed, the surgeon uses the suture eyelets to place a suture at either side of the device to prevent dislodgement of the device from the vagina in the post-operative period.

To assist with placement, the SVS device may include one or more resilient folding or bending features. A folding/bending feature may be in the form of a crease or cleft adapted to facilitate folding of the SVS device when a folding force is applied to the arched member, e.g. when opposing side portions of the arched member are squeezed together. Alternatively, the folding features may facilitate twisting or “rolling” of the SVS device to assist with placement by reducing the maximal transverse dimension (width) of the device as it is inserted into the vagina. The one or more folding features ideally have resilience so that the device resumes the substantially horse-shoe shape (i.e. unfolds) when the folding force is removed. In one embodiment, a folding feature is a cleft or crease provided substantially centrally of the arched member.

Ideally, the SVS device is substantially planar when viewed from its end or side. That is, the arched member, the opposing side portions and the transverse support member are provided in a common plane, as is the cross member when provided. The SVS device may be provided in a range of sizes to suit the anatomical features of different women treated using the device. Preferably, there are four sizes to choose from, although a choice as small as two may be sufficient and a range including 5 or 6 or more different sized SVS devices may be provided in some circumstances. Typically, size variation occurs in the length of the SVS device although less significant variations in the maximal transverse dimension are also possible when providing a range of differently sized SVS devices.

The length of a SVS device according to embodiments of the present invention may range between 40 mm and 100 mm although an length of between 55 mm and 85 mm is preferred. The maximal transverse dimension represents the “width” of the device at its widest part. Typically, this can be identified by locating the outermost parts of the opposing side portions of the arched member, before they become inwardly directed toward the proximally located transverse member. Ideally, the SVS device has a maximal transverse dimension of between 30 mm and 70 mm although a maximal transverse dimension of between 45 mm and 55 mm is preferred. The SVS device may have a transverse dimension in the vicinity of the cross member which is between 20 mm and 60 mm although transverse dimension of between 30 mm and 55 mm is preferred in that region.

Preferably, the arched member has a curved external profile adapted for atraumatic contact with the vaginal wall. The arched member may be circular in cross-section, or otherwise curved, such as a “D” or “C” shape, with the curved portion outwardly facing so as to minimise discomfort during post-surgical treatment using the SVS device. In some embodiments, the arched member of the SVS device has a thickness of between 6 mm and 15 mm and more preferably between 8 mm and 12 mm. Such thicknesses are thought to impart sufficient strength and rigidity to the device, whilst permitting folding for placement in the vagina with minimal discomfort for the patient.

It is to be understood that portions of the SVS device, including e.g. the arched member, may be formed from solid or hollow material, and may be porous or non-porous. In some embodiments the SVS device includes one or more openings or voids to reduce the amount of material required for manufacture. In other embodiments, the SVS device is made by moulding or extruding a deformable material such as medical-grade silicone or medical grade plastic/PVC.

Another aspect of the present invention provides a tamponading accessory configured for use with the SVS device described herein. The tamponading accessory includes an inflatable body having a substantially horse-shoe shape. Ideally, the inflatable body has an elongate dimension of approximately 100 mm and a maximal transverse dimension of approximately 70 mm with a transverse dimension at the base of the horse shoe shape of approximately 50 mm. Preferably, the tamponading accessory is also provided with inflation tubing adapted to couple with the inflatable body. The tubing may have a length of between 100 mm and 200 mm and is preferably 150 mm in length although the length may be trimmed prior to use.

Another aspect of the present invention provides a method for using a vaginal support device, such as the kind described above, to complement surgical treatment for pelvic organ prolapse. The method includes assembling the SVS device together with a tamponading balloon in an uninflated condition and, at the completion of surgical tissue repair, placing the SVS device and uninflated balloon in the vagina. The lateral aspects of the SVS device are attached to the vaginal wall and the balloon is inflated to achieve vaginal tamponading. After an initial period of between 20 and 36 hours and preferably approximately 24 hours post-surgery, the balloon is deflated and removed from the vagina while the SVS device remains in situ. After a tissue healing period of between 20 and 28 days after surgery, the SVS device is detached from the vaginal wall and removed from the vagina.

Typically, a syringe is used to inflate and deflate the balloon by coupling with inflation tubing connected to the tamponading balloon. The syringe is de-coupled from the inflation tubing during the initial period.

### BRIEF DESCRIPTION OF THE DRAWINGS

The present invention will now be described with reference to the embodiments shown in the accompanying drawings. It is to be understood that the embodiments described are provided by way of explanation only, and do not limit the scope of the invention as defined in the claims appended hereto.

FIG. 1 is a schematic illustration of a surgical vaginal support (SVS) device according to an embodiment of the invention, showing first end, top and second end views respectively.

FIG. 2 is a schematic illustration of the SVS device of FIG. 1 together with a tamponading accessory.

FIG. 3 is a schematic illustration of a tamponading accessory for use with the SVS device in FIGS. 1 and 2, together with a syringe for inflating the inflatable body of the tamponading accessory.

Table 1 shows suggested dimensions for SVS devices of four different sizes.

### DETAILED DESCRIPTION

Following conventional surgery for pelvic organ prolapse, the repaired tissues are exposed to increases in intra-abdominal pressure as the subject mobilizes, coughs, vomits, and strains with bowel evacuation. These rises in intra-abdominal pressure may adversely affect the healing of the vaginal repair and may lead to surgical failure and recurrent prolapse.

The present invention complements a new approach to surgical native tissue repair of pelvic organ prolapse and to sacral colpopexy and hysteropexy. At the completion of the surgical tissue repair phase, the inventive surgical vaginal support (SVS) device is placed into the vagina. The SVS device is used to support the healing vaginal tissues and remains in place for three to four weeks following surgery. By supporting the vagina with the SVS device for up to four weeks following surgery the risk of surgical failure and recurrent prolapse may be reduced.

An example of a SVS device according to the invention is illustrated in FIG. 1 which shows the distal end view, plan view and proximal end view, where the term “distal” refers to the end of the device adapted to rest toward the vaginal apex and “proximal” refers to the end of the device adapted to rest toward the vaginal opening when in use. The SVS device 100 has a substantially horse-shoe shape, with an arched member 110 that is curved distally, relative to a surgeon when placing the device. A transverse support member 120 is provided toward the proximal end of opposing side portions 112 of the arched member. The device is generally planar, with arched member 110, opposing side portions 112 and transverse support member provided in a common plane. In the embodiment illustrated, there is also cross member 130 connecting proximal ends of the side portions 112 of the arched member 110 also in the same plane. It is noted that the proximal end view (RHS of FIG. 1) does not show transverse member 120 behind cross member 130.

SVS device 100 has a substantially open structure. The inventor has discovered that an open structure, without a membrane or webbing connecting side portions 112 and/or distal arched aspect of arched member 110 may lead to a reduction in post-operative discharge. Thus, the SVS device is frame-like in structure, providing a scaffolding to support post-surgical healing of repaired tissue. The scaffolding comprises an arched member which, in a preferred embodiment, has a substantially circular cross section with a diameter of approximately 6 mm to 15 mm and more preferably between 8 mm and 12 mm.

Other cross sectional profiles are contemplated for the arched member 110, such e.g. as D-shaped or C-shaped, where the curved edge is outwardly facing as shown in the end views of FIG. 1, to make atraumatic contact with the vaginal wall when the SVS device is in situ. The arched member 110 may be solid, hollow, porous or non-porous and may be adapted to elute therapeutic agents to assist tissue healing and mitigate infection. One or more openings may be provided along a length of the arched member 110 and there may be an inwardly or outwardly directed channel or slot for receiving part of an inflatable body of a tamponading accessory with which the SVS device may be used. Receiving part of the inflatable body in this way may assist with placement of the SVS device together with the inflatable body, prior to inflation.

The transverse support member 120 maintains ideal separation between the side portions 112 of arched member 110 and imparts strength to the SVS device 100 as well as assisting to maintain the generally horse shoe shape. It is desirable that the arched member 110 (and ideally the totality of the SVS device 100) is manufactured from a semi-rigid biocompatible material that has sufficient stiffness to maintain the horseshoe shape and support the vaginal wall during a period of 20 to 28 days while tissue healing occurs, without causing discomfort. The material should also be non-reactive (e.g. medical grade silicone). In a preferred embodiment, the material also permits folding or rolling of the SVS device, as may be required for initial placement into the vagina.

In the embodiment illustrated, a folding feature 150 in the form of a cleft or crease is provided substantially centrally of the arched member 110. Folding feature 150 aids folding of the SVS device 100 by urging the device to fold or flex inwardly when a folding force is applied. Typically a folding force is applied by the surgeon squeezing the opposing side portions 112 of the arched member 110 together. When the folding force is removed (e.g. when the SVS device 100 is placed into the vagina) folding feature 150 permits the SVS device to resume a substantially horse-shoe shape.

Folding, rolling or twisting the SVS device 100 may assist with placement by temporarily reducing the maximal transverse dimension of the device.

Ideally, the SVS device is available in more than one size, to accommodate different female anatomies, typically determined by the length of the vagina. The surgeon may gauge the vaginal length at completion of the tissue repair phase of the surgery with a ruler. Thus, the size of the SVS device used will typically depend on the length of the repaired vagina in each case.

Ideally, the SVS device 100 is provided in at least four sizes: small, medium, large and extra-large. Length dimensions of the SVS device typically range from 55 mm to 85 mm although longer lengths (such as e.g. 110 mm) are also contemplated. Maximal transverse dimensions of the SVS device, i.e. in the region of the broadest portion of the arched member 100, typically range from 45 mm to 55 mm although dimensions of e.g. 30 to 70 mm are contemplated. Transverse dimensions of the SVS device in the region of cross member 130 may range from e.g. 30 mm to 55 mm although dimensions of between 20 mm and 60 mm are contemplated.

Table 1 illustrates dimensions that are suggested for SVS devices of four different sizes identified as small (S), medium (M), large (L) and extra-large (XL). It is to be noted that the relationship between length and maximal transverse dimension (width) is not constant between sizes. The variance in the transverse dimension is slight compared to the variance in length. Different sizes of SVS devices may be utilised for different surgeries for repairing pelvic organ prolapse. For instance, the XL device may be preferred for robotic and laparoscopic surgeries, and open colpopexy.

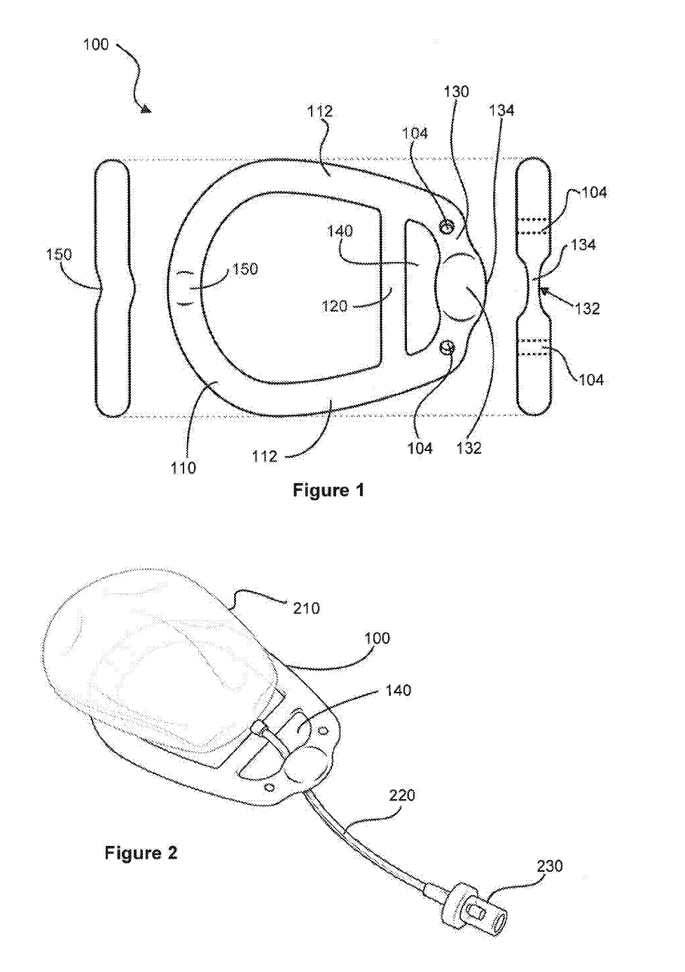

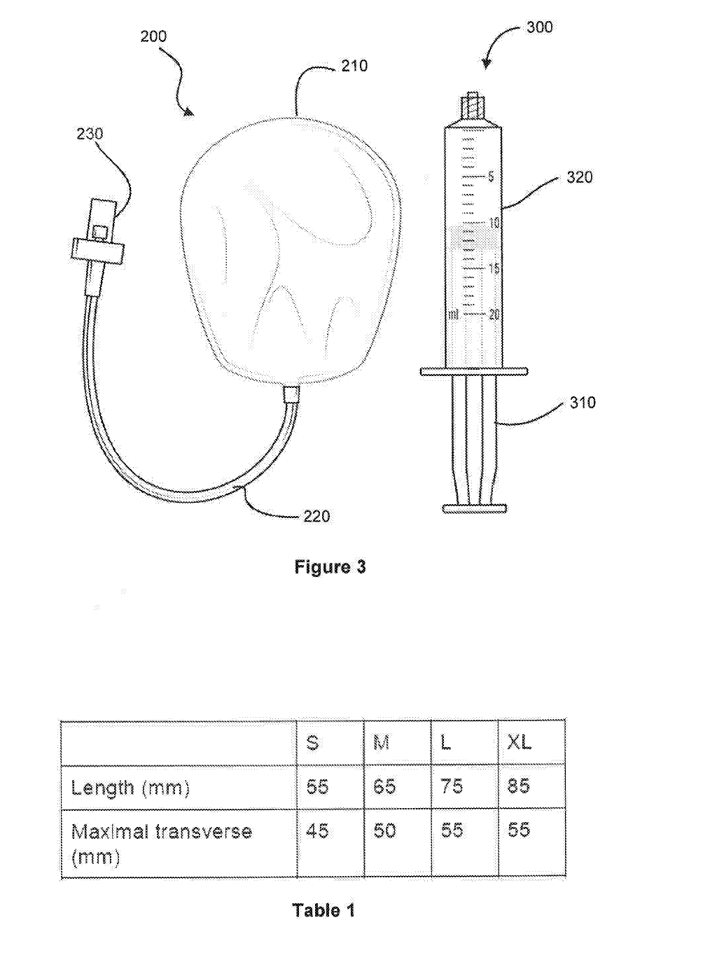

Preferably, the SVS device 100 is used together with a tamponading accessory 200 incorporating an inflatable body 210 in the form of a balloon (FIGS. 2 and 3). The inflatable balloon 210 of tamponading accessory 200 has a substantially horse-shoe shape to provide effective tamponading of repaired tissue when the balloon is inflated, including in the vicinity of the vaginal apex. Typically, inflatable balloon 210 has an elongate dimension of approximately 100 mm, a maximal transverse dimension of approximately 70 mm and minimal transverse dimension (toward the base of the horse shoe) of approximately 50 mm although longer and wider balloons are also contemplated. For some patients, it may be desirable for the balloon to have shorter and/or narrower dimensions, as may be ascertained by one of skill in the art.

An appropriately sized SVS device 100 is coupled with the tamponading accessory 200 by passing the inflation tubing 220 through an opening 140 between transverse support member 120 and cross member 130 of the SVS device. When tubing 220 is threaded through opening 140, the inflatable body 210 is placed over the SVS device so that the respective horse shoe shapes of the SVS device 100 and the inflatable balloon 210 are substantially aligned. Alternatively/additionally, a distal portion of the uninflated balloon 210 may be folded over the distally arched end of the SVS device 100 prior to insertion into the vagina. Folding the distal portion of the inflatable balloon 210 in this way may assist with maintaining alignment between the balloon and the SVS device 100 as they are placed into the vagina and during inflation of the balloon.

The SVS device 100 and uninflated balloon 210 of tamponading accessory 200 are placed in the vagina at the completion of the tissue repair phase of the surgery for pelvic organ prolapse. When appropriately placed, the SVS device 100 is attached to the vaginal wall and the balloon 210 is inflated. Once inflated, balloon 210 of the tamponading accessory 200 is used instead of a vaginal pack to tamponade the vagina to reduce postoperative venous bleeding. Correct placement is with the proximal end of the SVS device (containing the suture eyelets) just above the hymen.

Eyelets 104 are provided on each side of SVS device 100, at the lateral aspects of the cross member 130. When appropriately placed, the SVS device 100 is temporarily attached to the vaginal wall using sutures placed through eyelets 104 to prevent dislodgement. It is to be understood, however, that different fasteners such as tacks or the like may be used to prevent dislodgement of the device as the patient convalesces.

Inflation tubing 220 provides fluid coupling between inflatable balloon 210 and a syringe 320 (FIG. 3) or other source of inflation fluid. Ideally, inflation tubing 220 is between 100 mm and 200 mm in length. For most surgeries, a length of about 150 mm is considered ideal. Preferably, balloon 210 is inflated with air, although other fluid (e.g. saline) could be used. Inflation tubing 220 is coupled with syringe 320 using syringe coupling 330 and air is pushed into the tubing 220, to inflate balloon 210 by pushing plunger 310 into barrel 320.

The proximal end of inflation tubing 220 has a coupling 230 to accommodate syringe 300. Fluid is passed through tubing 220 to allow inflation and deflation of the inflatable balloon 210. It is to be understood, however, that other fluid sources such as low pressure pumps may be utilised to inflate the balloon. A cap, valve or crimping device is ideally located near the proximal end of the inflation tubing 220 to minimise unwanted escape of fluid from the inflatable balloon 210 and the tubing, while the tamponading accessory 200 is being used. The inflatable balloon 210 is deflated and retracted through opening 124 in SVS device 100 and removed, approximately 24 hours following surgery. This involves re-coupling syringe 300 with inflation tubing 220 and releasing the cap, valve or crimping device so that fluid may be withdrawn from balloon 210 causing it to deflate. Since the inflatable balloon 210 is deflated prior to removal of the tamponading accessory 200, it is easily withdrawn from the vagina without dislodging the SVS device 100, and with minimal discomfort to the patient.

The tamponading accessory 200 or a component thereof (such as an inflation balloon 210 and/or inflation tube 220) may be provided in conjunction with the SVS device 100, e.g. in a kit form. Alternatively, the SVS device 100 may be provided separately from the tamponading accessory 200. Inflatable body 210 of tamponading accessory 200 is ideally manufactured from soft and expandable, biocompatible, non-reactive material such as e.g. medical grade silicone or PVC.

After removal of tamponading accessory 200, the SVS device 100 remains in situ for three to four weeks. Approximately 25 to 28 days after the surgery the sutures are removed from suture eyelets 104 and SVS device 100 is removed. In the SVS device 100 of FIGS. 1 and 2, cross member 130 includes a region 132 of reduced thickness for grasping during removal of the SVS device from the vagina. Thinner portion 132 of cross member 130 provides an easily locatable portion for the surgeon to grasp for removal. Thinner region 132 may include a lobe 134 extending proximally, which the surgeon may grasp. A grasping device may also be used to withdraw the SVS device 100 by grasping or coupling with cross member 130 at lobe 134. Preferably, lobe 134 is integral with the cross member 130. Although removal of the SVS device 100 first requires removal of the sutures or other fasteners holding it in place, advantageously this can be performed in the consulting room; readmission to hospital is generally not required.

Conventional vaginal surgery (native tissue repair) for pelvic organ prolapse carries with it a significant risk of recurrent prolapse following initial surgery. Using the inventive SVS device to support the healing vaginal tissues following surgery is a novel approach for native tissue repair for pelvic organ prolapse. Use of the SVS device for three to four weeks following surgery may reduce the risk of prolapse recurrence without an increased risk of morbidity when compared to native tissue repair for pelvic organ prolapse without a SVS device.

The inflatable tamponading accessory and particularly, the inflatable tamponading balloon, replaces traditional gauze packing to achieve haemostasis immediately following surgery. Use of the inflatable tamponading accessory post-operatively is believed to cause less post-operative discomfort than the traditional gauze pack. Removal is achieved by deflating the tamponading balloon, and withdrawing the balloon from the SVS device and vagina. Because the balloon is deflated, it has little or no effect on the placement of the SVS device, and causes less discomfort during removal than gauze packs or other traditional tamponading accessories.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present invention as defined in the provisional claims appended hereto.

It is to be understood that the following claims are provided by way of example only. Features may be added to or omitted from the claims at a later date so as to further define or re-define the invention or inventions.

1. A surgical vaginal support (SVS) device for use with surgical treatment of pelvic organ prolapse, the SVS device having a substantially horse-shoe shape, including an arched member which is curved distally and a transverse support member connecting opposing side portions of the arched member toward a proximal end thereof.

2. A SVS device according to claim 1 further including a cross member connecting proximal ends of the arched member side portions.

3. A SVS device according to claim 2, wherein the cross member includes a region of reduced thickness for grasping during removal of the SVS device from the vagina.

4. A SVS device according to claim 3 including a lobe for grasping during removal of the SVS device from the vagina, the lobe extending proximally of the cross member.

5. A SVS device according to claim 4 wherein the lobe is integral with the cross member.

6. A SVS device according to claim 1, including an opening, defined between the transverse support member and the cross member, for removably receiving part of a tamponading accessory.

7. A SVS device according to claim 1, including one or more fastening features for removably attaching the SVS device to the vaginal wall.

8. A SVS device according to claim 7 wherein the one or more fastening features include one or more suture eyelets.

9. A SVS device according to claim 1, including one or more resilient folding features adapted to facilitate folding of the SVS device when a folding force is applied to the arched member, and to resume the substantially horse-shoe shape when the folding force is removed.

10. A SVS device according to claim 9 wherein one of the one or more folding features is provided substantially centrally of the arched member.

11. A SVS device according to claim 1, wherein the arched member and the opposing side portions and the transverse support member are provided in a common plane.

12. A SVS device according to claim 1, selected from a plurality of SVS devices provided in a range of different sizes.

13. A SVS device according to claim 1, wherein the SVS device has an elongate dimension of between 40 mm and 100 mm and preferably between 55 mm and 85 mm.

14. A SVS device according to claim 1, wherein the SVS device has a maximal transverse dimension of between 30 mm and 70 mm and preferably between 45 mm and 55 mm.

15. A SVS device according to claim 2, wherein the SVS device has a transverse dimension in the vicinity of the cross member which is between 20 mm and 60 mm and preferably between 30 mm and 55 mm.

16. A SVS device according to claim 1, wherein the arched member has a thickness of between 6 mm and 15 mm and preferably between 8 mm and 12 mm.

17. A SVS device according to claim 1, wherein the arched member has a curved external profile adapted for atraumatic contact with the vaginal wall.

18. A tamponading accessory configured for use with the SVS device according to claim, the tamponading accessory including an inflatable body having a substantially horse-shoe shape.

19. A tamponading accessory according to claim 18, wherein the inflatable body has an elongate dimension of approximately 100 mm.

20. A tamponading accessory according to claim 18, wherein the inflatable body has a maximal transverse dimension of approximately 70 mm.

21. A tamponading accessory according to claim 18, wherein the inflatable portion has a transverse dimension at a base of the horse shoe shape of approximately 50 mm.

22. A tamponading accessory according to claim 18, including inflation tubing couplable with the inflatable body and having a length of between 100 mm and 200 mm, preferably 150 mm.

23. A SVS device according to claim 1, provided in conjunction with a tamponading accessory.

24. A method of using a surgical vaginal support (SVS) device as an adjunct to surgical treatment for pelvic organ prolapse, the method including the steps of:
(a) providing the SVS device with a tamponading balloon in an uninflated condition;
(b) after surgical repair of tissue, placing the SVS device and uninflated balloon in the vagina;
(c) attaching lateral aspects of the SVS device to the vaginal wall;
(d)inflating the tamponading balloon to achieve vaginal tamponading;
(e) after an initial period, deflating the tamponading balloon and removing the balloon from the vagina; and
(f) after a tissue healing period, detaching the SVS device from the vaginal wall and removing the SVS device from the vagina;
wherein the initial period is between 20 and 36 hours and is approximately 24 hours after completion of surgery, and wherein the tissue healing period is between 20 and 28 days after completion of surgery.

25. A method of using a SVS device according to claim 24 wherein the SVS device has a substantially horse-shoe shape, including an arched member which is curved distally and a transverse support member connecting opposing side portions of the arched member toward a proximal end thereof.

26. A method of using a vaginal support device according to claim 24, wherein the SVS device is attached by placing sutures through suture eyelets in lateral aspects of a cross member of the SVS device.

27. A method according to claim 24, wherein the assembling step includes receiving inflation tubing attached to the tamponading balloon through an opening in the SVS device.

28. A method according to claim 24, including the step of coupling a syringe with inflation tubing attached to the tamponading balloon to inflate and deflate the balloon, wherein the syringe is de-coupled from the inflation tube during the initial period.
